(19) United States
(12) Patent Application Publication
(10) Pub. No.: US 2016/0206296 A1
(43) Pub. Date: Jul. 21, 2016

(54) Biopsy Syringe Device

(71) Applicant: Hamid Ehsani-Nia, Northridge, CA (US)

(72) Inventor: Hamid Ehsani-Nia, Northridge, CA (US)

(21) Appl. No.: 15/080,761

(22) Filed: Mar. 25, 2016

(51) Int. Cl. A61B 10/02; A61M 1/00

(52) U.S. Cl. CPC A61B 10/0283; A61M 1/0062; A61M 2005/3201

(57) ABSTRACT

A biopsy device including a biopsy needle having an interior lumen and an exterior surface and a secondary solution sleeve comprising an interior cylindrical portion and an exterior cylindrical portion disposed about the interior cylindrical portion and concentric thereto defining a lumen between the interior and exterior cylindrical portions, wherein the biopsy needle is sized to fit within the interior cylindrical portion and is concentric therewith, and wherein following obtaining of a biopsy specimen, the biopsy needle is segregated from healthy tissue by enclosure of the biopsy needle in the sleeve.

### FIELD OF THE DISCLOSED TECHNOLOGY

The disclosed technology relates generally to a biopsy device and, more specifically, to a biopsy device which ensures that the biopsy needle and specimen are segregated from the surrounding healthy tissue and portions of the device which engage such surrounding tissue.

### BACKGROUND OF THE DISCLOSED TECHNOLOGY

Biopsies of tissue which is suspected to be diseased or to require treatment is a commonly used diagnostic procedure. Typically, a biopsy needle is inserted into the tissue to be biopsied, and a specimen of the tissue is drawn into the needle. The needle is then retracted, through healthy tissue, in order to remove the needle from the body. In some cases, prior to retraction of the needle, a secondary substance, such as a bio-adhesive or a pharmaceutical is injected into the biopsy site.

One major disadvantage of the currently used methodology is that insertion of the biopsy needle into the biopsied tissue results in contamination of the needle, and following retraction of the needle through healthy tissue can result in contamination of the healthy tissue. For example, removal of the contaminated biopsy syringe or biopsy needle through healthy tissue can result in spreading of infection, spreading of degrading enzymes or digestive juices, exposing immunologically isolated regions of the body to the immune system, or seeding cancerous cells in healthy regions of the body.

U.S. Pat. No. 6,702,760 to Krause et al discloses a biopsy device with coagulant. This patent is concerned with collection of tissue specimens and includes a proximal end with application channel and a distal end with a wider channel. A tube or needle is movable in and out of the application channel to take a biopsy, while fluids can be applied through the wider application channel. Retraction of the needle causes material to be forced out of the outer tube. However, retraction of the biopsy needle into the device results in contamination of the fluids or secondary solution by the wall of the biopsy needle due to contact therebetween. Thus, the secondary solution being injected is no longer considered sterile, because it has been contaminated by whatever substance(s) are disposed on the biopsy needle following its penetration into the biopsy site.

U.S. Pat. No. 8,486,155 to McAlister et al discloses a fistula repair plug having multiple layers. A surgical device is disclosed to plug within a lumen of a patient. Media from previous chambers can be expelled into the patient. It appears that the cartridges used to house liquids using for plugging are movable on support rails. However, the disclosed device does not include a biopsy needle, and the application needles do not include any needles positioned one within another.

U.S. Pat. No. 5,325,857 to Nabai et al discloses a skin biopsy device using a syringe. A cylindrical plug is cut from a pad of an absorbable sponge and is implanted in a biopsy site. The two step process includes first taking a biopsy and removing the syringe, and then using the same syringe as it is the same size of the hole and punching out a hold of sponge and depositing it in the biopsy site. As such, the contaminated syringe is introduced into healthy tissue again following removal of the biopsy specimen.

Therefore, there exists a need in the art for a biopsy needle device including a biopsy needle and a lumen for application of a secondary solution, which are independently movable relative to one another, such that contaminated material, such as the biopsy needle following obtaining of the biopsy specimen, is not exposed at any stage to healthy tissue or to the secondary solution injected into the healthy tissue.

### SUMMARY OF THE DISCLOSED TECHNOLOGY

The disclosed technology relates generally to biopsy devices and, more specifically, to a biopsy device which ensures that the biopsy needle and specimen are segregated from the surrounding healthy tissue and portions of the device which engage such surrounding tissue.

There is thus provided, in accordance with an embodiment of the disclosed technology, a biopsy device, including:

a biopsy needle having an interior lumen adapted to have disposed therein a biopsy specimen, and an exterior surface;

a biopsy syringe fluidly connected to the biopsy needle and including a syringe piston, the biopsy syringe adapted to receive the biopsy specimen from the biopsy needle;

a first motor functionally associated with the biopsy needle and driving longitudinal movement of the biopsy needle along a longitudinal axis thereof;

a secondary solution sleeve including an interior cylindrical portion and an exterior cylindrical portion disposed about the interior cylindrical portion and concentric thereto defining a lumen between the interior and exterior cylindrical portions; and

a secondary solution syringe, fluidly connected to the secondary solution sleeve, the secondary solution syringe adapted to contain a secondary solution designed to be injected into an injection site via the secondary solution sleeve;

wherein the biopsy needle is sized to fit within the interior cylindrical portion, is concentric therewith, and is retractable into an interior of the interior cylindrical portion under the force of the first motor, such that the biopsy needle and the interior lumen thereof are segregated from the lumen by the interior cylindrical portion.

In some embodiments, the biopsy needle is longer than the secondary solution sleeve.

In some embodiments, the exterior cylindrical portion of the secondary solution sleeve includes at an end thereof at least one slot in fluid communication with the lumen, the at least one slot adapted to have the secondary solution exit the lumen therethrough.

In some embodiments the biopsy device further includes a first piston motor functionally associated with the syringe piston and adapted to drive longitudinal movement of the syringe piston.

In some embodiments, the secondary solution syringe includes a secondary solution piston adapted to propel the secondary solution toward the secondary solution sleeve, the device further including a second piston motor functionally associated with the secondary solution piston and adapted to drive longitudinal movement thereof.

In some embodiments, the first motor includes at least one longitudinally extending rail, the biopsy syringe is mounted on the at least one rail and supported thereby such that motion of the biopsy syringe is limited to a single dimension. In some such embodiments, the biopsy syringe is removable from the rails.

In some embodiments, the secondary solution syringe is removable from the device for replacement thereof or for filling thereof.

There is further provided in accordance with another embodiment of the present invention a method for obtaining a biopsy specimen, the method including: providing a biopsy device as described hereinabove; positioning the biopsy device such that the biopsy needle and the secondary solution sleeve are adjacent a boundary of a biopsy site; operating the first motor to propel the biopsy needle into the biopsy site; withdrawing a biopsy specimen from the biopsy site into the biopsy syringe via the biopsy needle; injecting a secondary solution from the secondary solution sleeve to the vicinity of the biopsy site around the needle and a lesion caused thereby; operating the first motor to retract the needle, through the injected secondary solution, into the sleeve; and withdrawing the needle and the sleeve out of the body via healthy tissue surrounding the biopsy site, wherein the exterior surface of the needle is segregated from the healthy tissue by enclosure thereof in the sleeve.

In some embodiments, the secondary solution includes at least one component which, when the secondary solution is injected to the vicinity of the biopsy site, forms a physical barrier separating the biopsy needle from healthy tissue therearound. In some embodiments, the at least one component includes at least one of fibringlue and at least one coagulant. In some embodiments, the secondary solution includes at least one therapeutic component for treating the biopsy site. In some embodiments, the secondary solution includes at least one prevention component adapted to prevent damage to healthy tissue surrounding the biopsy site by contaminants from the biopsy site.

There is additionally provided in accordance with yet another embodiment of the disclosed technology a method for obtaining a biopsy specimen, the method including providing a biopsy device including a biopsy needle having an interior lumen and an exterior surface and a secondary solution sleeve including an interior cylindrical portion and an exterior cylindrical portion disposed about the interior cylindrical portion and concentric thereto defining a lumen between the interior and exterior cylindrical portions, wherein the biopsy needle is sized to fit within the interior cylindrical portion and is concentric therewith. Further steps can or do include positioning the biopsy device such that the biopsy needle and the sleeve are adjacent a boundary of a biopsy site; propelling the biopsy needle into the biopsy site; withdrawing a biopsy specimen from the biopsy site into the biopsy needle; retracting the needle from the biopsy site into the interior cylindrical portion of the sleeve; and withdrawing the needle and the sleeve out of the body via healthy tissue surrounding the biopsy site, wherein the exterior surface of the needle is segregated from the healthy tissue by enclosure thereof in the sleeve.

In some embodiments, the lumen of the sleeve is fluidly connected to at least one secondary solution reservoir and the exterior cylindrical portion of the sleeve includes at least one slot, and the method further includes, while the sleeve is positioned adjacent the boundary of the biopsy site, injecting a secondary solution from the secondary solution reservoir to the boundary of the biopsy site, via the at least one slot in the sleeve.

In some embodiments, the secondary solution includes at least one component which, when the secondary solution is injected to the vicinity of the biopsy site, forms a physical barrier separating the biopsy needle from healthy tissue there-around. In some embodiments, the at least one component includes at least one of fibringlue and at least one coagulant. In some embodiments, the secondary solution includes at least one therapeutic component for treating the biopsy site. In some embodiments, the secondary solution includes at least one prevention component adapted to prevent damage to healthy tissue surrounding the biopsy site by contaminants from the biopsy site.

### BRIEF DESCRIPTION OF THE DRAWINGS

FIGS. 1A and 1B show a perspective view illustration and a side view planar illustration of a biopsy device, according to an embodiment of the disclosed technology.

FIGS. 2A and 2B show a front perspective view illustration and a side view planar illustration of a biopsy needle and a secondary solution sleeve forming part of the biopsy device of FIGS. 1A and 1B, according to an embodiment of the disclosed technology.

FIG. 3 shows a rear perspective view illustration of the biopsy device of FIGS. 1A and 1B, according to an embodiment of the disclosed technology.

FIGS. 4A and 4B show two alternate embodiments of a secondary solution sleeve according to embodiments of the disclosed technology.

FIG. 5 shows a side plan view illustration of the biopsy needle and secondary solution sleeve of the biopsy device of FIGS. 1A and 1B according to embodiments of the disclosed technology, and corresponding cross sectional illustrations taken at various locations along the biopsy needle and secondary solution sleeve.

FIGS. 6A and 6B show a side view planar illustration of a biopsy device according to embodiments of the disclosed technology in a preparation stage, and an enlargement of a portion thereof including the biopsy needle and the secondary solution sleeve.

FIGS. 7A and 7B show a side view planar illustration of a biopsy device according to embodiments of the disclosed technology in a body wall penetration stage, and an enlargement of a portion thereof including the biopsy needle and the secondary solution sleeve.

FIGS. 8A and 8B show a side view planar illustration of a biopsy device according to embodiments of the disclosed technology in a biopsy needle insertion stage, and an enlargement of a portion thereof including the biopsy needle and the secondary solution sleeve.

FIG. 9 shows a side view planar illustration of a biopsy device according to embodiments of the disclosed technology in a specimen extraction stage.

FIGS. 10A and 10B show a side view planar illustration of a biopsy device according to embodiments of the disclosed technology in a secondary solution injection stage, and an enlargement of a portion thereof including the biopsy needle and the secondary solution sleeve.

FIGS. 11A and 11B show a side view planar illustration of a biopsy device according to embodiments of the disclosed technology in a biopsy needle retraction stage, and an enlargement of a portion thereof including the biopsy needle and the secondary solution sleeve.

FIG. 12 shows a side view planar illustration of a biopsy device according to embodiments of the disclosed technology in a biopsy device extraction stage.

### DETAILED DESCRIPTION OF EMBODIMENTS OF THE DISCLOSED TECHNOLOGY

In an embodiment of the disclosed technology, a biopsy device includes a biopsy syringe and needle for collecting a biopsy specimen, and a secondary solution syringe and sleeve for delivering a secondary solution to the biopsy site. The biopsy syringe is fully retractable into the secondary solution sleeve, such that the secondary solution and healthy tissue surrounding the biopsy site are fully and completely segregated from the biopsy needle and specimen, which may carry contaminants from the biopsy site.

It is appreciated that in the context of the description and claims herein, the terms “forward”, “anterior”, “protecting” and equivalents thereof relates to the needle end of the biopsy device or to motion toward the needle end of the biopsy device or toward a target tissue. Similarly, the terms “backward”, “rearward”, “posterior”, “retracting” and equivalents thereof relate to the plunger end of the biopsy device, or to motion away from the needle end of the biopsy device or away from a target tissue.

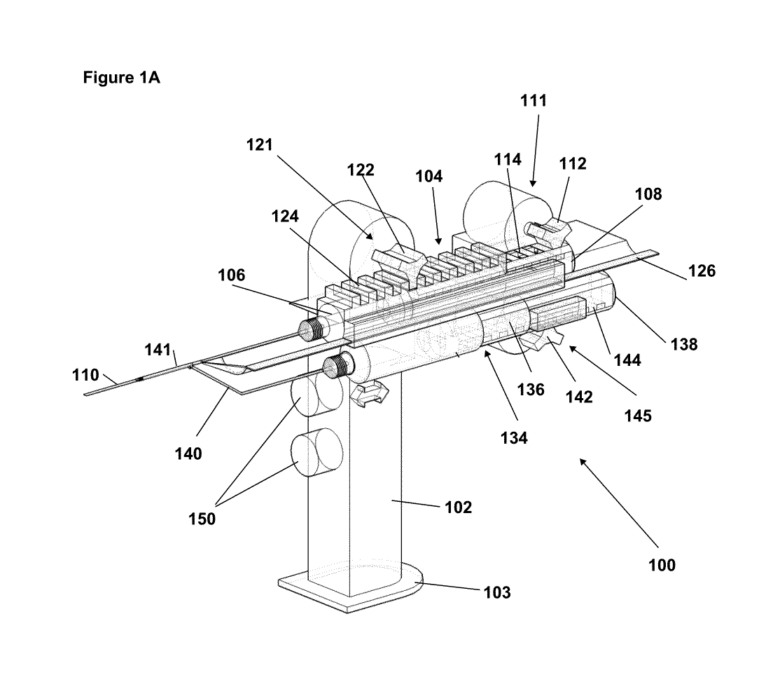

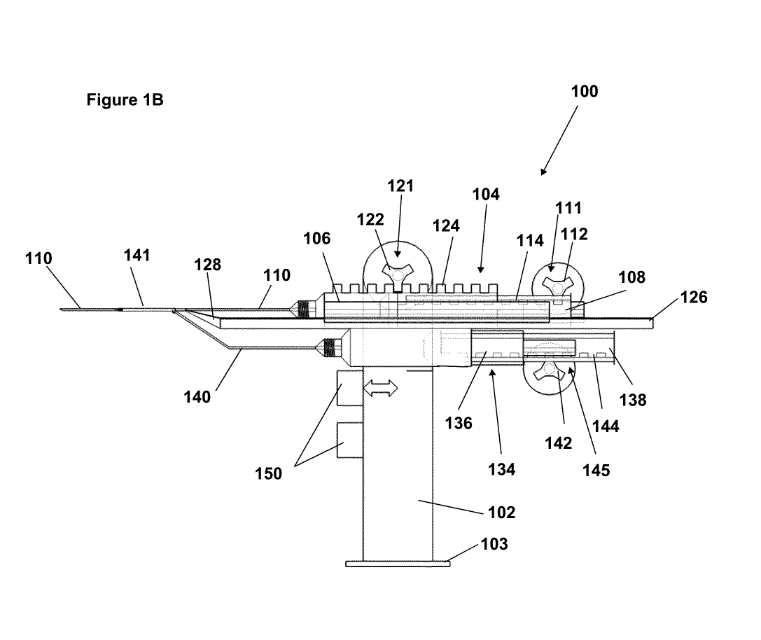

Reference is now made to FIGS. 1A and 1B, which show a perspective view illustration and a side view planar illustration of a biopsy device 100, according to an embodiment of the disclosed technology. As seen, the biopsy device 100 includes a base 102 which in some embodiments can stand stably on a suitable base platform 103. The base 102 has mounted thereon a biopsy arrangement 104 including a biopsy syringe 106. The biopsy syringe 106 includes a piston 108 and terminates in a biopsy needle 110 which is in fluid flow communication with a specimen collection chamber of syringe 106, such that there is continuous fluid flow communication between the lumen of biopsy needle 110 and the specimen collection chamber, or lumen, of syringe 106. As explained in further detail hereinbelow, the needle 110 includes a cylindrical wall, defining a longitudinal lumen for collecting a biopsy specimen therein.

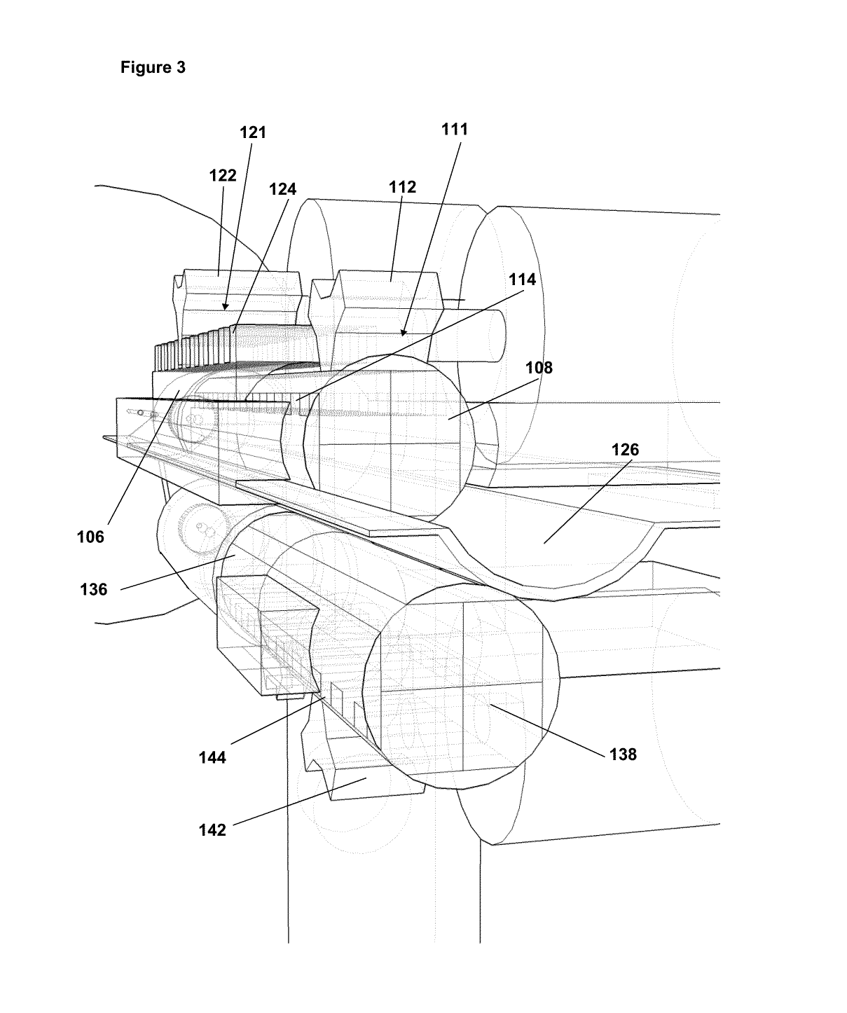

Reference is additionally made to FIG. 3, which shows a rear perspective view illustration of the biopsy device 100. As seen in FIGS. 1A, 1B, and 3, the piston 108 may be functionally associated with a biopsy piston motor 111, adapted to move the piston 108 along a longitudinal axis thereof, such that rearward motion of piston 108 under the driving force of the piston motor causes the biopsy specimen to be drawn into biopsy needle 110, as explained hereinbelow. In the illustrated embodiment, the biopsy piston motor 111 is a rack and pinion mechanism and includes a cog or pinion 112 functionally associated with a rack 114 connected to piston 108, such that rotation of cog 112 results in longitudinal motion of rail 114 together with the piston 108, as known in the art of rack and pinion motors. It is appreciated that operation of the biopsy piston motor may be pre programmed, or may be affected by the user actively initiating such operation, for example by providing electronic instructions via a computer, pushing a suitable button 150 as described hereinbelow, or operating a suitable lever.

The biopsy arrangement 104, including the biopsy syringe 106, piston 108, biopsy needle 110, and the biopsy piston motor 111, are functionally associated with a biopsy motor 121 adapted to move the entire biopsy arrangement 104 along a longitudinal axis of biopsy syringe 106, thereby to move the biopsy syringe 106 and biopsy needle 110 forward or rearward relative to the base 102, as explained hereinbelow. In the illustrated embodiment, the biopsy motor is a rack and pinion mechanism and includes a cog or pinion 122 functionally associated with a rack 124 connected to biopsy syringe 106, such that rotation of cog 122 results in longitudinal motion of rack 124 together with the biopsy arrangement 104 as known in the art. In some embodiments the biopsy syringe 106, piston 108, and biopsy needle 110 are removably connected to rail 124, and are removable therefrom for extraction of a biopsy specimen and/or for replacement or sterilization of the syringe 106, piston 108 and needle 110 following contamination thereof, for example after a biopsy procedure is complete. It is appreciated that operation of the biopsy motor may be pre programmed, or may be affected by the user actively initiating such operation, for example by providing electronic instructions via a computer, pushing a suitable button or operating a suitable lever.

As seen with particular clarity in FIG. 3, in some embodiments the biopsy arrangement 104 is disposed on a base tray 126 extending laterally from base 102, which supports the biopsy syringe 106 and ensures alignment of the piston 108 with the piston motor and of the biopsy arrangement 104 with the biopsy motor (defined as a mechanism to cause movement of the biopsy arrangement). In some embodiments the base tray 126 includes at least one curved portion, conforming in shape to the shape of biopsy syringe 106. Additionally, the base tray 126 may provide a stabilizing and/or juxtaposing force to the biopsy piston motor 111 and pinion 112 thereof, and to biopsy motor 121 and pinion 122 thereof. The base tray may also include an inclined portion 128, supporting biopsy needle 110 and providing stability thereto.

Returning to FIGS. 1A and 1B, it is seen that base 102 has mounted thereon a secondary solution arrangement 134, which may be disposed beneath the biopsy arrangement 104 and which includes a secondary solution syringe 136. The secondary solution syringe 136 includes a piston 138 and terminates in a conduit 140 which is in fluid flow communication with a chamber of syringe 136. As explained in further detail hereinbelow, the conduit 140 is in fluid flow communication with a secondary solution sleeve 141, adapted to be positioned around needle 110.

The piston 138 may be functionally associated with a second piston motor 145 adapted to move the piston 138 along a longitudinal axis thereof, such that forward motion of piston 138 under the driving force of the piston motor causes the secondary substance in the chamber of secondary substance syringe 136 to be propelled into sleeve 141 via conduit 140, as explained hereinbelow. In the illustrated embodiment, the second piston motor 145 is a rack and pinion mechanism and includes a pinion or cog 142 functionally associated with a rack 144 connected to piston 138, such that rotation of cog 142 results in longitudinal motion of rack 144 together with the piston 138. It is appreciated that operation of the second piston motor 145 may be pre programmed, or may be affected by the user actively initiating such operation, for example by providing electronic instructions via a computer, pushing a suitable button or operating a suitable lever.

In some embodiments, the secondary solution arrangement 134 is removable from base 102, for example for filling thereof or for replacement of components thereof, as necessary.

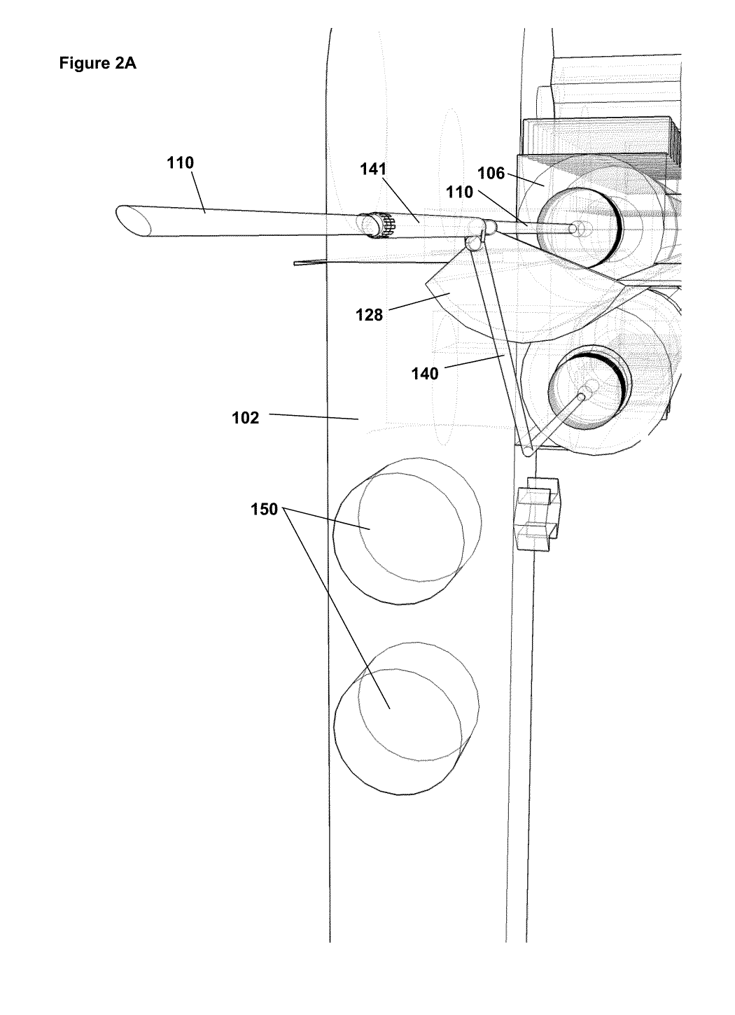

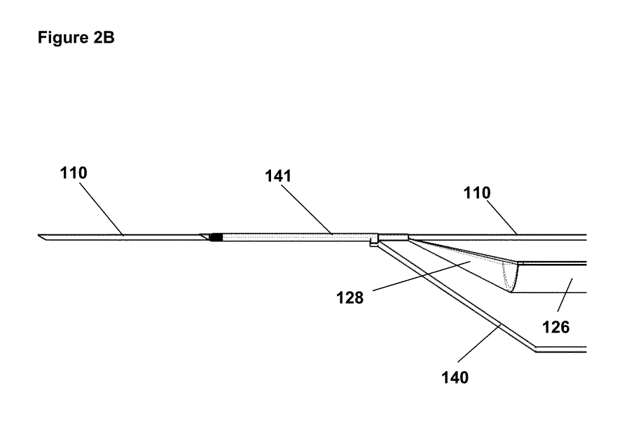

Reference is now additionally made to FIGS. 2A and 2B, which show a front perspective view illustration and a side view planar illustration of biopsy needle 110 and secondary solution sleeve 141, according to an embodiment of the disclosed technology. As seen, conduit 140 extends from secondary solution syringe 136 to secondary solution sleeve 141, which sleeve is disposed about biopsy needle 110 but is shorter than the biopsy needle, such that the sleeve 141 surrounds only a portion of needle 110. It is a particular feature of the disclosed technology that needle 110 is longitudinally movable within sleeve 141, and is arranged such that in some situations a contaminated portion of needle 110 is within sleeve 141, such that any contaminants present on and/or within the needle 110 are segregated from the surrounding areas traversed by the device.

Returning to FIGS. 1A and 1B, in some embodiments the base 102 includes one or more buttons 150 for controlling one or more of the first piston motor, the second piston motor, and the biopsy motor. In other embodiments, the motors and the device 100 may include a processing unit programmed to automatically activate the motors at suitable times and to provide suitable motion so as to perform a biopsy procedure as explained hereinbelow. In some such embodiments, the processing unit is functionally associated with a transitive, and may receive input and/or instructions from a remote location, such a computer or hand held computing device operated by a physician.

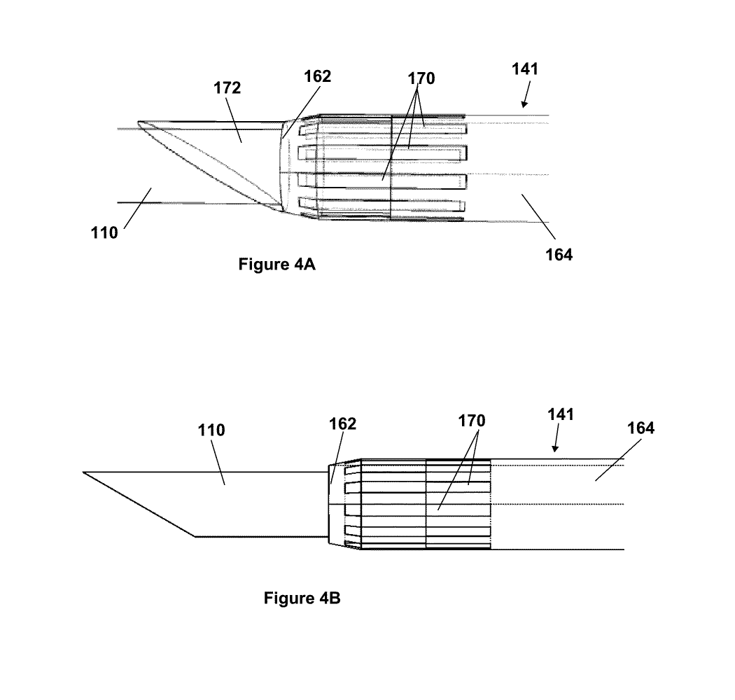

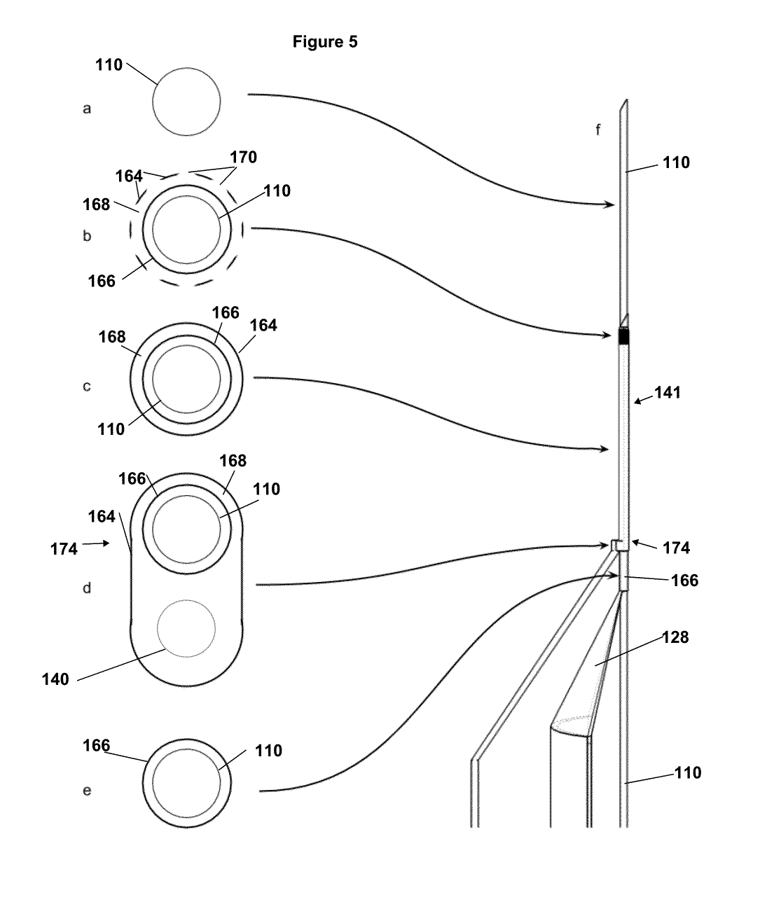

Reference is now made to FIGS. 4A and 4B, which show two alternate embodiments of secondary solution sleeve 141 according to embodiments of the disclosed technology, as well as to FIG. 5, which shows a side plan view illustration of the biopsy needle 110 and secondary solution sleeve 141 and corresponding cross sectional illustrations taken at various locations along the biopsy needle and secondary solution sleeve.

As seen in FIGS. 4A and 4B, secondary solution sleeve 141 includes a main body portion having an anterior end 162. As clear from cross sectional illustration c in FIG. 5, sleeve 141 includes an exterior cylindrical portion 164 having an interior cylindrical portion 166 disposed therein and concentric thereto, such that the exterior and interior cylindrical portions 164 and 166 connect to one another at anterior end 162, such that at the anterior portion thereof the outer cylindrical portion 164 tapers and becomes narrower, until a having a diameter equal or close to that of cylindrical portion 166 at end 162 . A lumen 168 is formed between the cylindrical portions 164 and 166, and terminates at anterior end 162. Exterior cylindrical portion 164 includes a plurality of slots or fenestration 170 adapted for passage of a secondary solution therethrough. In some embodiments, the plurality of slots 170 are formed as a mesh, so as to prevent excessive damage to tissues traversed by sleeve 141 during the biopsy procedure.

As explained in further detail hereinbelow, during use of the device 100, a secondary solution flows is propelled by piston 138 from secondary solution syringe 136, via conduit 140 to lumen 168, and flow out of slots 170 to an injection site. It is a particular feature of the disclosed technology that, as seen in sectional illustration d of FIG. 5, interior cylindrical portion 166 does not include any slots corresponding to slots 170, and thus when the needle 110 situated within inner cylindrical portion 166 of sleeve 141 is contaminated, the contaminants thereon are fully segregated from the secondary solution disposed in lumen 168 of sleeve 141.

Turning specifically to FIG. 4A, it is seen that in some embodiments the secondary solution sleeve 141 includes a beveled tip portion 172 located distally to anterior end 162, which tip portion corresponds in alignment to, and can be considered a continuation of, interior cylindrical portion 166. In some embodiments, the angle of tip portion 172 may correspond to an angle of the tip of needle 110. The tip portion 172 is obviated in the embodiment of FIG. 4B, in which the sleeve 141 terminates anterior end 162.

In some embodiments, the interior cylindrical portion 166 extends proximally beyond the end of exterior cylindrical portion 164, as seen in cross sectional illustration e in FIG. 5. In some such embodiments, interior cylindrical portion 166 may extend all the way to inclined surface 128, as shown in the main portion of FIG. 5.

As seen in FIG. 5 and in sectional illustration d thereof, a joint portion 174 fluidly connects conduit 140 with lumen 168 of secondary solution sleeve 141, by providing a short segment of exterior cylindrical portion having a large enough circumference to encircle interior cylindrical portion 166 as well as an end of conduit 140.

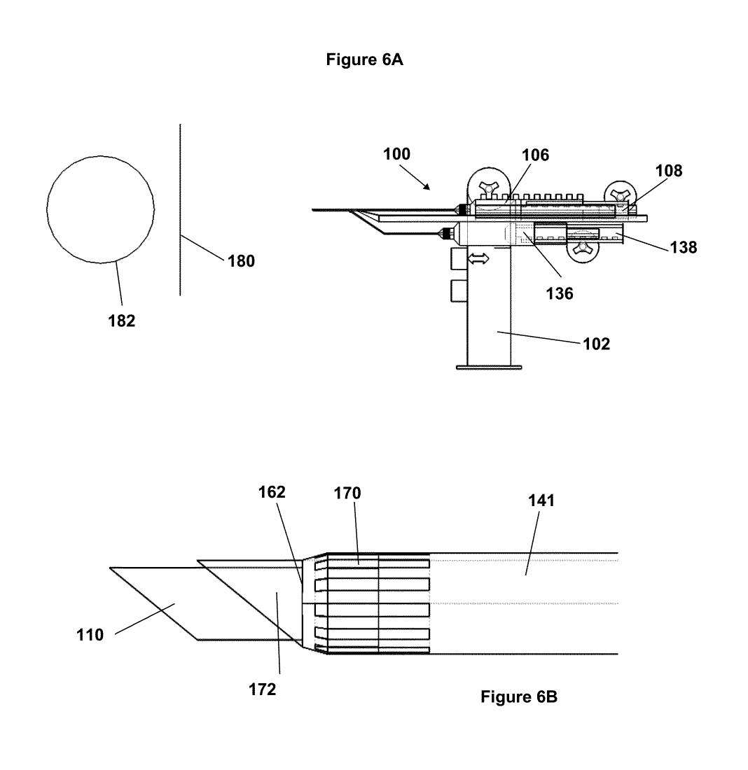

FIGS. 6A to 12, described hereinbelow, illustrate steps of a method for obtaining a biopsy specimen and injecting a secondary solution to the biopsy site using the device 100 described hereinabove.

Reference is now made to FIGS. 6A and 6B, which show a side view planar illustration of biopsy device 100 in a preparation stage, and an enlargement of a portion thereof including biopsy needle 110 and secondary solution sleeve 141.

As seen in FIG. 6A, the device 100 is distant from the body wall 180, such as the skin or any other body layer or barrier to be penetrated, and is positioned such that if the needle 110 were moved forward it would reach a biopsy site 182 located beyond the body wall. Piston 138 is posteriorly drawn, due to the secondary solution which is contained in secondary solution syringe 136, whereas piston 108 of biopsy syringe 106 is in its most anterior position, so as to be retractable when the needle 110 is located in the biopsy site. The biopsy needle 110 protrudes slightly from secondary solution sleeve 141, and the angle of tip portion 172 is aligned with the angle of needle 110, as seen clearly in FIG. 6B.

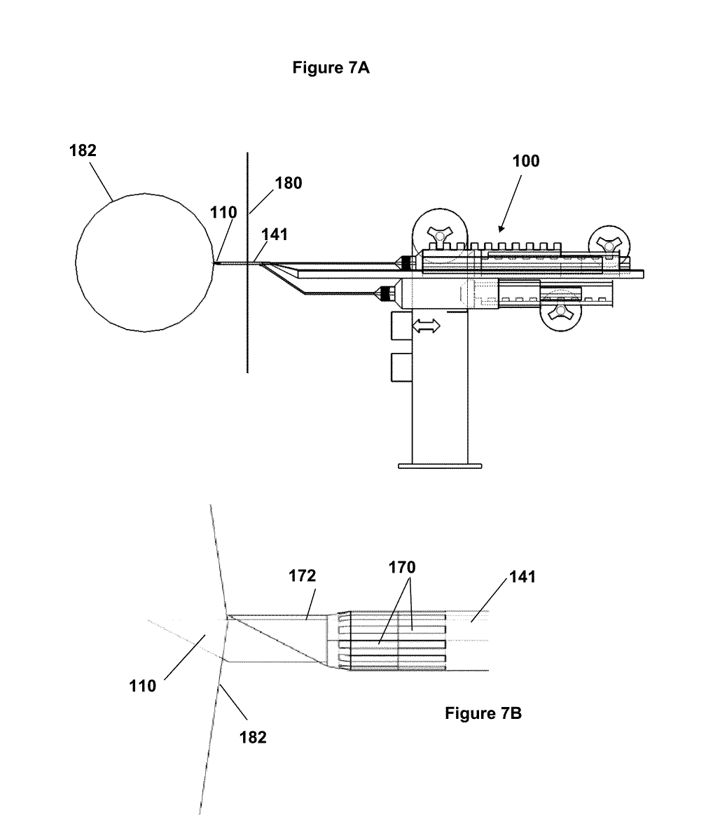

Turning now to FIGS. 7A and 7B, which show a side view planar illustration of biopsy device 100 in a body wall penetration stage and an enlargement of a portion thereof, it is seen that the device 100 is moved, for example by manipulation of base 102, toward the body wall 180, such that needle 110 and sleeve 141 penetrate the body wall 180. The needle 110 also slightly penetrates into biopsy site 182, while the tip 172 of sleeve 141 is disposed just outside the biopsy site and adjacent a boundary thereof, and is surrounded by healthy tissue, as seen clearly in FIG. 7B. It is appreciated that slots 170, formed in sleeve 141, prevent excessive damage from being incurred to the healthy tissue disposed between the body wall 180 and the biopsy site 182 during penetration of needle 110 and sleeve 141 into the body for example due to the mechanics of reduced drag.

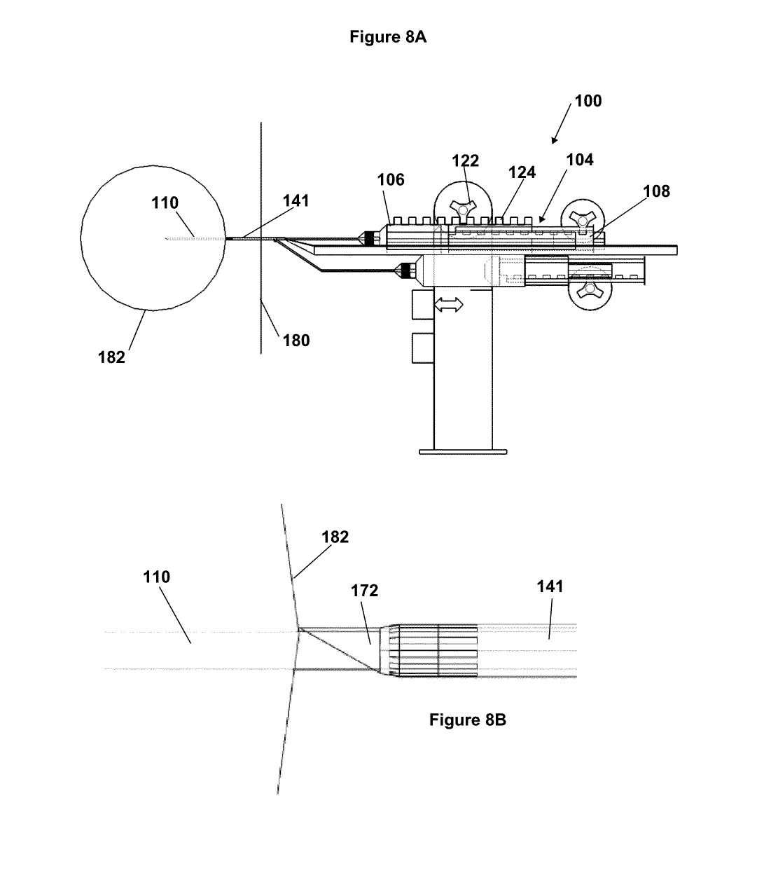

Reference is now made to FIGS. 8A and 8B, which show a side view planar illustration of biopsy device 100 in a biopsy needle insertion stage, and an enlargement of a portion thereof including biopsy needle 110 and secondary solution sleeve 141. As seen in FIG. 8A, the entire biopsy arrangement 104, including the biopsy syringe 106, piston 108, biopsy needle 110, and the piston motor have been displaced toward the body wall 180 under the force of the biopsy motor, by rotation of cog 122 which results in displacement of rack 124 and the arrangement 104 carried thereby. The remainder of device 100, including base 102, secondary solution arrangement 134, and specifically sleeve 141, remain stationary. It is appreciated that the motion of the biopsy arrangement 104 is isolated in one dimension (the longitudinal direction of the biopsy syringe 106), due to the stability of motion and support of weight provided by the rack 124, in addition to the biopsy arrangement 104 gliding on rails defined by lateral edges of the tray 126.

The forward motion of the biopsy arrangement 104 causes biopsy needle 110 to extend further into the biopsy site 182, as seen clearly in FIG. 8B. However, it is a particular feature of the disclosed technology that sleeve 141 remains stationary at the edge of the biopsy site, continues to be surrounded by healthy tissue, and is segregated from contaminants of the biopsy site 182.

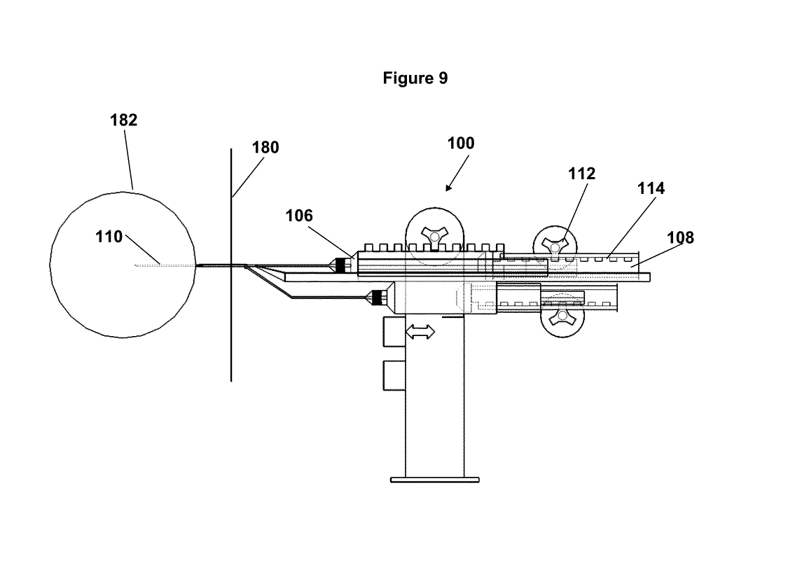

Turning now to FIG. 9, which shows a side view planar illustration of biopsy device 100 in a specimen extraction stage, it is seen that the piston 108 of biopsy syringe 106 is retracted under the force of the piston motor, and specifically due to rotation of cog 112 relative to rack 114. The retraction of piston 108 results in a biopsy specimen being drawn from the biopsy site 182, into needle 110. At the end of the sampling process, the specimen may reside in the lumen of needle 110, or may move therefrom into the chamber of biopsy syringe 106, depending on the extent to which the piston 108 was drawn. All other components of the device 100 remain stationary.

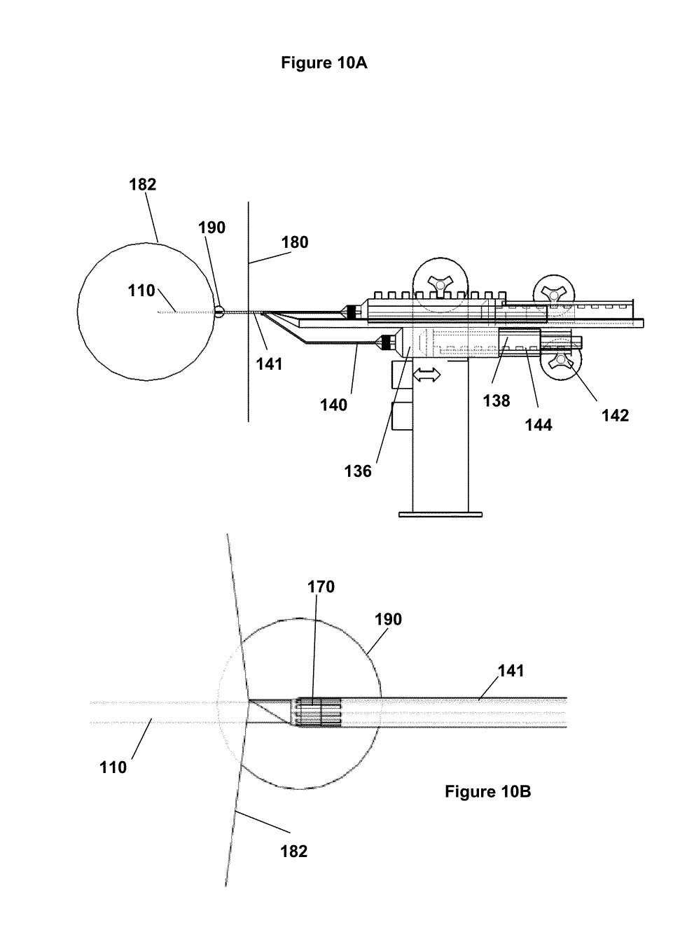

Reference is now made to FIGS. 10A and 10B, which show a side view planar illustration of biopsy device 100 in a secondary solution injection stage, and an enlargement of a portion thereof. As seen, following extraction of the biopsy specimen, a secondary solution is injected from secondary solution syringe 136, via conduit 140, secondary solution sleeve 141, and slots or fenestration 170 thereof, into the healthy tissue surrounding the site at which biopsy needle 110 entered the biopsy site 182. The injected secondary solution is indicated by reference numeral 190. Injection of the secondary solution is affected by forward motion of piston 138 relative to secondary solution syringe 136, under the driving force of the second piston motor and specifically due to rotation of cog 142 relative to rack 144.

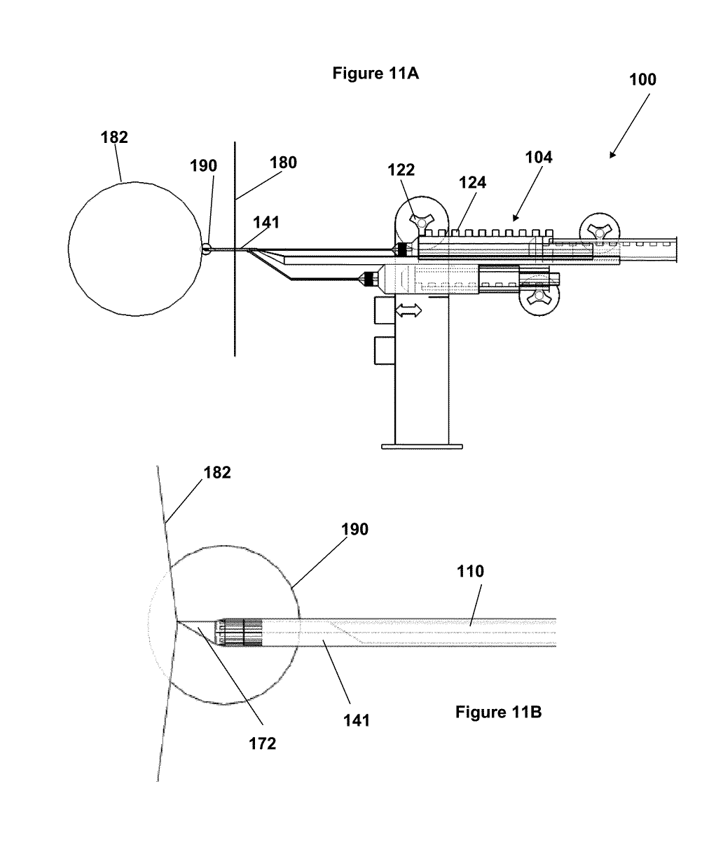

The secondary solution injected into the healthy tissue adjacent the biopsy site is designed to form a physical barrier separating the lesion formed by the biopsy needle 110 in the biopsy site 182 from the healthy tissue surrounding the biopsy site, as well as to enclose the needle 110 and segregate it from the surrounding healthy tissue while it is being retracted from the biopsy site 182, as explained in further detail hereinbelow with reference to FIGS. 11A and 11B, which illustrate the next step of the process. Thus, as seen clearly in FIG. 10B, the secondary solution 190 surrounds the tip 172 of sleeve 141 as well as the portion of needle 110 exposed between the sleeve 141 and the biopsy site 182. In some embodiments, the secondary solution includes a temporary or a permanent glue, such a fibringlue, and may additionally include clotting or coagulation factors, all of which support the formation of a physical barrier between healthy tissue and the contaminated lesion of the biopsy site and/or the now contaminated biopsy needle 110. The injected secondary solution 190 is capable of forming a physical plug or seal at the biopsy site that has been lesion ed by the biopsy needle 110

In some embodiments, the secondary solution may further include components specifically targeted to the biopsy site and designed to assist in treatment or healing thereof. For example, in cases in which the biopsy site is an abscess, the secondary solution may include antibiotics for treatment of the infection in the abscess. Similarly, in cases in which the biopsy site is in a digestive organ the secondary substance may include one or more components for neutralizing digestive enzymes. As another examples, if the biopsy site is a cancerous tumor, the secondary solution may include radioisotopes, chemotherapeutic agents, and/or molecules that specifically target the specific cell surface marker of that tumor.

Turning now to FIGS. 11A and 11B, which show a side view planar illustration of biopsy device 100 in a biopsy needle retraction stage, and an enlargement of a portion thereof, it is seen that biopsy needle 110 is retracted from the biopsy site 182 and into the secondary solution sleeve 141, until the section of the needle which engaged biopsy site 182 is fully enclosed in the sleeve 141. Such retraction is accomplished by the entire biopsy arrangement 104 being longitudinally posteriorly displaced relative to the biopsy site 182 under the driving force of the biopsy motor, and specifically due to rotation of cog 122 relative to rack 124. As discussed hereinabove, the motion of biopsy arrangement 104 is one-dimensional, due to the support provided by rack 124 and by the rails formed by the lateral edges of tray 126.

It is a particular feature of the disclosed technology that during retraction thereof, the needle 110, which is now contaminated by contaminants from the biopsy site, is fully segregated from the healthy tissue surrounding the biopsy site. Specifically, the secondary solution sleeve 141 is disposed immediately adjacent the boundary of biopsy site 182, and as such each retracting section of the needle is immediately covered by the sleeve 141. The short segment of the biopsy needle 110 which may be exposed to the surrounding healthy tissue during retraction thereof due to the geometries of the needle, the sleeve, and the biopsy site boundary, is segregated from the surrounding tissue by the physical barrier formed by the injected secondary solution 190. As such, any contamination of the healthy tissue surrounding the biopsy site, such as spreading of infection from an abscess biopsy site or seeding cancerous cells picked up by the needle during biopsy of a tumor, is prevented.

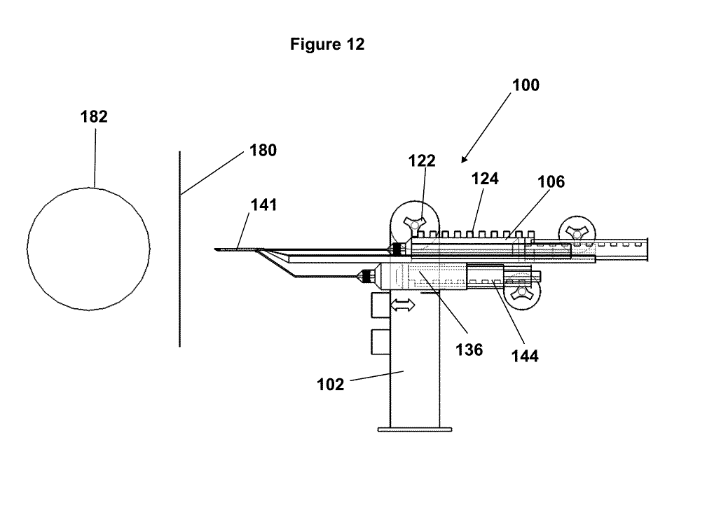

Reference is finally made to FIG. 12, which shows a side view planar illustration of biopsy device 100 in a biopsy device extraction stage. As seen, the biopsy device is moved away from the body 180, for example by a user holding base 102, such that sleeve 141 is retracted from the body and exits the body wall 180. Following removal of the device 100 from the body, the biopsy device 100 may be taken apart to prepare it for subsequent use and to analyze the contents thereof. For example, syringe 106 may be removed from rack 124 and from the rails of tray 126 for analysis of the biopsy specimen contained therein, syringe 136 may be removed from rack 144 for refilling or replacement with another syringe, and sleeve 141 and needle 110, which engaged portions of the body, may be removed from the device 100 for sterilization or for replacement with a new, sterile counterparts.

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalency of the claims are to be embraced within their scope. Combinations of any of the methods and apparatuses described hereinabove are also contemplated and within the scope of the invention.

1. A biopsy device, comprising:
a biopsy needle having an interior lumen adapted to have disposed therein a biopsy specimen, and an exterior surface;
a biopsy syringe fluidly connected to said biopsy needle and including a syringe piston, the biopsy syringe adapted to receive said biopsy specimen from said biopsy needle;
a first motor functionally associated with said biopsy needle and driving longitudinal movement of said biopsy needle along a longitudinal axis thereof;
a secondary solution sleeve comprising an interior cylindrical portion and an exterior cylindrical portion disposed about said interior cylindrical portion and concentric thereto defining a lumen between said interior and exterior cylindrical portions; and
a secondary solution syringe, fluidly connected to said secondary solution sleeve, said secondary solution syringe adapted to contain a secondary solution designed to be injected into an injection site via said secondary solution sleeve;
wherein said biopsy needle is sized to fit within said interior cylindrical portion, is concentric therewith, and is retractable into an interior of said interior cylindrical portion under the force of said first motor, such that said biopsy needle and said interior lumen thereof are segregated from said lumen by said interior cylindrical portion.

2. The biopsy device of claim 1, wherein said biopsy needle is longer than said secondary solution sleeve.

3. The biopsy device of claim 1, wherein said exterior cylindrical portion of said secondary solution sleeve includes at an end thereof at least one slot in fluid communication with said lumen, said at least one slot adapted to have said secondary solution exit said lumen there-through.

4. The biopsy device of claim 1, further comprising a first piston motor functionally associated with said syringe piston and adapted to drive longitudinal movement of said syringe piston.

5. The biopsy device of claim 1, wherein said secondary solution syringe includes a secondary solution piston adapted to propel said secondary solution toward said secondary solution sleeve, the device further comprising a second piston motor functionally associated with said secondary solution piston and adapted to drive longitudinal movement thereof.

6. The device of claim 1, wherein said first motor comprises at least one longitudinally extending rail, said biopsy syringe is mounted on said at least one rail and supported thereby such that motion of said biopsy syringe is limited to a single dimension.

7. The device of claim 6, wherein said biopsy syringe is removable from said rails.

8. The device of claim 1, wherein said secondary solution syringe is removable from said device for replacement thereof or for filling thereof.

9. A method for obtaining a biopsy specimen, the method comprising:
providing a biopsy device according to claim 1;
positioning said biopsy device such that said biopsy needle and said secondary solution sleeve are adjacent a boundary of a biopsy site;
operating said first motor to propel said biopsy needle into said biopsy site;
withdrawing a biopsy specimen from said biopsy site into said biopsy syringe via said biopsy needle;
injecting a secondary solution from said secondary solution sleeve to the vicinity of said biopsy site around said needle and a lesion caused thereby;
operating said first motor to retract said needle, through said injected secondary solution, into said sleeve; and
withdrawing said needle and said sleeve out of the body via healthy tissue surrounding said biopsy site, wherein said exterior surface of said needle is segregated from said healthy tissue by enclosure thereof in said sleeve.

10. The method of claim 9, wherein said secondary solution includes at least one component which, when the secondary solution is injected to the vicinity of said biopsy site, forms a physical barrier separating said biopsy needle from healthy tissue therearound.

11. The method of claim 10, wherein said at least one component comprises at least one of fibringlue and at least one coagulant.

12. The method of claim 9, wherein said secondary solution includes at least one therapeutic component for treating said biopsy site.

13. The method of claim 9, wherein said secondary solution includes at least one prevention component adapted to prevent damage to healthy tissue surrounding said biopsy site by contaminants from said biopsy site.

14. A method for obtaining a biopsy specimen, the method comprising:
providing a biopsy device including:
a biopsy needle having an interior lumen and an exterior surface; and
a secondary solution sleeve comprising an interior cylindrical portion and an exterior cylindrical portion disposed about said interior cylindrical portion and concentric thereto defining a lumen between said interior and exterior cylindrical portions;
wherein said biopsy needle is sized to fit within said interior cylindrical portion and is concentric therewith;
positioning said biopsy device such that said biopsy needle and said sleeve are adjacent a boundary of a biopsy site;
propelling said biopsy needle into said biopsy site;
withdrawing a biopsy specimen from said biopsy site into said biopsy needle;
retracting said needle from said biopsy site into said interior cylindrical portion of said sleeve; and
withdrawing said needle and said sleeve out of the body via healthy tissue surrounding said biopsy site, wherein said exterior surface of said needle is segregated from said healthy tissue by enclosure thereof in said sleeve.

15. The method of claim 14, wherein said lumen of said sleeve is fluidly connected to at least one secondary solution reservoir and said exterior cylindrical portion of said sleeve includes at least one slot, the method further comprising:
while said sleeve is positioned adjacent said boundary of said biopsy site, injecting a secondary solution from said secondary solution reservoir to said boundary of said biopsy site, via said at least one slot in said sleeve.

16. The method of claim 14, wherein said secondary solution includes at least one component which, when the secondary solution is injected to the vicinity of said biopsy site, forms a physical barrier separating said biopsy needle from healthy tissue therearound.

17. The method of claim 16, wherein said at least one component comprises at least one of fibringlue and at least one coagulant.

18. The method of claim 14, wherein said secondary solution includes at least one therapeutic component for treating said biopsy site.

19. The method of claim 14, wherein said secondary solution includes at least one prevention component adapted to prevent damage to healthy tissue surrounding said biopsy site by contaminants from said biopsy site.
